(19) United States
(12) Patent Application Publication
(10) Pub. No.: US 2026/0130672 A1
(43) Pub. Date: May 14, 2026

(54) VESSEL BELT

(71) Applicants: Pusan National University Industry-University Cooperation Foundation, Busan (KR); Pusan National University Hospital, Busan (KR)

(72) Inventors: Sangsu LEE, Yangsan-si (KR); Junyong JEKAL, Yangsan-si (KR)

(73) Assignees: Pusan National University Industry-University Cooperation Foundation, Busan (KR); Pusan National University Hospital, Busan (KR)

(21) Appl. No.: 19/389,495

(22) Filed: Nov. 14, 2025

(30) Foreign Application Priority Data
Nov. 14, 2024 (KR) 10-2024-0162095

(51) Int. Cl. A61B 17/132; A61B 17/11

(52) U.S. Cl. CPC A61B 17/1327; A61B 2017/1107

(57) ABSTRACT

A vessel belt includes a body portion in the form of a band that wraps around a blood vessel, a plurality of engaging protrusions arranged along a longitudinal direction on an outer surface of the body portion, and a fixing wire having both ends fixed to opposite sides of a rear end of the body portion, the fixing wire being disposed at a predetermined distance from the outer surface of the body portion such that a front end of the body portion and the engaging protrusions pass thereunder, and after the front end of the body portion has passed, a rear portion of one of the plurality of engaging protrusions is caught and supported by the fixing wire.

### CROSS-REFERENCE TO RELATED PATENT APPLICATION

This application claims priority to Korean Patent Application No. 10-2024-0162095 (filed on Nov. 14, 2024), which is all hereby incorporated by reference in its entirety.

### BACKGROUND

The present disclosure relates to a vessel belt that wraps around a blood vessel, and more specifically, to a vessel belt that wraps around an outer surface of a blood vessel in the form of a band to suppress expansion of the blood vessel.

### 2. DESCRIPTION OF THE RELATED ART

After a period of time following the creation of an arteriovenous fistula for dialysis, the vein connected to the artery may become excessively large in diameter, causing excessive blood flow and increasing the burden on the heart, which may negatively affect the patient's long-term prognosis and cause various problems, such as the need to create a new dialysis blood vessel (this is called High Flow Access (HFA)).

In order to overcome this situation, there are various surgical methods to limit the size of blood vessels, but all existing techniques for limiting blood vessel size are complicated or burdensome to the patient.

### PRIOR-ART DOCUMENTS

### Patent Documents

(Patent Document 1) International Patent Publication No. WO 2005-041781 A1 (published on May 12, 2005)

(Patent Document 2) Korean Patent No. 10-2628297 (registered on Jan. 18, 2024)

### SUMMARY

The present disclosure is intended to solve the above problem, and an object of the present disclosure is to provide a vessel belt that is able to be mounted around of a blood vessel in the form of a band with a simple operation, and be easily adjusted in diameter according to the diameter of the blood vessel while being fastened around the blood vessel to effectively suppress expansion of the blood vessel.

In order to achieve the above object, a vessel belt according to the present disclosure includes a body portion in the form of a band that wraps around a blood vessel, a plurality of engaging protrusions arranged along a longitudinal direction on an outer surface of the body portion, and a fixing wire having both ends fixed to opposite sides of a rear end of the body portion, the fixing wire being disposed at a predetermined distance from the outer surface of the body portion such that a front end of the body portion and the engaging protrusions pass thereunder, and after the front end of the body portion has passed, a rear portion of one of the plurality of engaging protrusions is caught and supported by the fixing wire.

Two wing portions may be formed to laterally protrude from opposite side edges of the rear end of the body portion, and both ends of the fixing wire may be inserted into and fixed to the respective wing portions.

The fixing wire may be made of titanium.

The front end of the body portion may be shaped to be pointed forward.

A through hole for tool insertion may be formed in the front end of the body portion.

Each engaging protrusion may be formed in a ratchet shape having a curved front surface, and the engaging protrusions may be arranged at intervals equal to the diameter of the fixing wire.

A thickness of the body portion may be formed to gradually decrease toward the front.

A vessel belt according to another aspect of the present disclosure includes a body portion made of a resin material in the form of a band that wraps around a blood vessel and has a front end formed to be pointed forward, a plurality of engaging protrusions arranged along a longitudinal direction on an outer surface of the body portion, each engaging protrusion being formed in a ratchet shape having a curved front surface, two wing portions formed to laterally protrude from opposite side edges of a rear end of the body portion, and a fixing wire having both ends fixed to the wing portions at opposite sides of the rear end of the body portion, the fixing wire being disposed at a predetermined distance from the outer surface of the body portion so that the front end of the body portion and the engaging protrusions pass under the fixing wire, and after the front end of the body portion has passed, a rear portion of one of the plurality of engaging protrusions is caught and supported by the fixing wire.

The fixing wire may be made of titanium.

A through hole for tool insertion may be formed in the front end of the body portion.

According to the present disclosure, a body portion in the form of a band is able to be rolled into a circle while passing through the lower side of a fixing wire to wrap around a blood vessel, and when it stops moving at a diameter corresponding to the size of the blood vessel, the circle is able to be maintained, so that the procedure becomes very simple and easy, and there is an advantage in that the diameter is able to be adjusted as desired according to the size of the blood vessel.

### BRIEF DESCRIPTION OF THE DRAWINGS

FIG. 1 is a perspective view showing a vessel belt according to an embodiment of the present disclosure.

FIG. 2 is an enlarged perspective view of a portion of the vessel belt illustrated in FIG. 1.

FIG. 3 is a perspective view showing a state of use of the vessel belt illustrated in FIG. 1.

FIG. 4 is a cross-sectional view showing a state of use of the vessel belt illustrated in FIG. 1.

### DETAILED DESCRIPTION

A vessel belt according to embodiments of the present disclosure will be described in detail with reference to the attached drawings. The present disclosure may be modified in various ways and may take many forms, and specific embodiments are illustrated in the drawings and described in detail in the text. However, this is not intended to limit the present disclosure to a specific disclosure form, and it should be understood that it includes all modifications, equivalents, or substitutes included in the spirit and technical scope of the present disclosure. In describing each drawing, similar reference numerals are used for similar components. In the accompanying drawings, the dimensions of structures are shown enlarged from the actual size to ensure clarity of the present disclosure, or reduced from the actual size to understand the schematic configuration.

Additionally, although terms such as first and second may be used to describe various components, the components should not be limited by the terms. The terms are used solely to distinguish one component from another. For example, without departing from the scope of the present disclosure, a first component may be referred to as a second component, and similarly, the second component may also be referred to as the first component. Unless otherwise defined, all terms used herein, including technical or scientific terms, have the same meaning as commonly understood by a person skilled in the art to which the present disclosure pertains. Terms defined in commonly used dictionaries should be construed to have meaning consistent with their meaning in the context of the relevant technology, and will not be construed in an idealized or overly formal sense unless expressly defined in the present application.

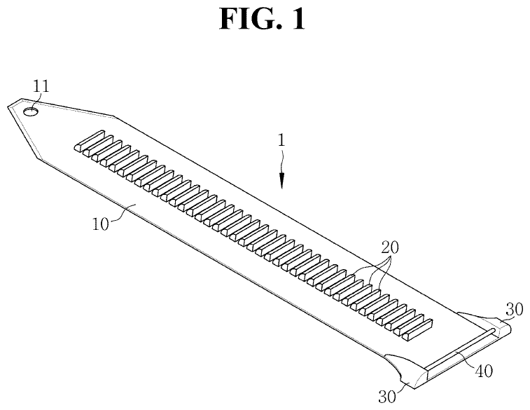

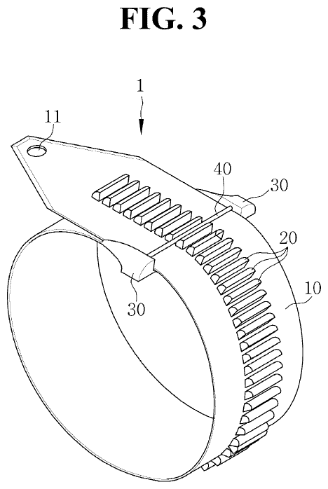

FIGS. 1 to 4 illustrate a vessel belt 1 according to an embodiment of the present disclosure, and the vessel belt 1 may include a body portion 10, a plurality of engaging protrusions 20, a wing portion 30, and a fixing wire 40.

The body portion 10 is formed in a band shape and made of a flexible resin material so as to wrap around a blood vessel, and a front end of the body portion 10 may have a pointed shape directed forward so as to easily pass under the fixing wire 40. The body portion 10 may be made of a resin material such as polyurethane that is harmless to the human body and flexible.

The thickness of the body portion 10 is preferably approximately 0.15 mm, which provides appropriate strength and resilience and does not cause discomfort inside the human body. In order to prevent damage to the blood vessel when the body portion 10 passes under the blood vessel, the thickness of the body portion 10 may be formed to become thinner as it goes forward.

In addition, in order to enable the front end of the body portion 10 to be easily pulled using a tool so that the body portion 10 passes under the blood vessel, a through hole 11 for tool insertion may be formed through the front end of the body portion 10. The through hole 11 may be formed in a circular shape so that the tool may be easily engaged therewith and it is less likely to tear when pulled by the tool.

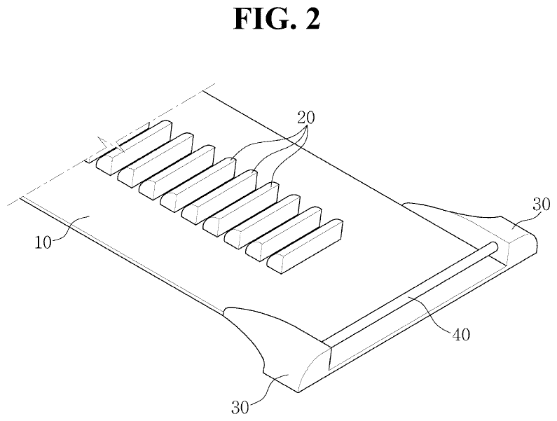

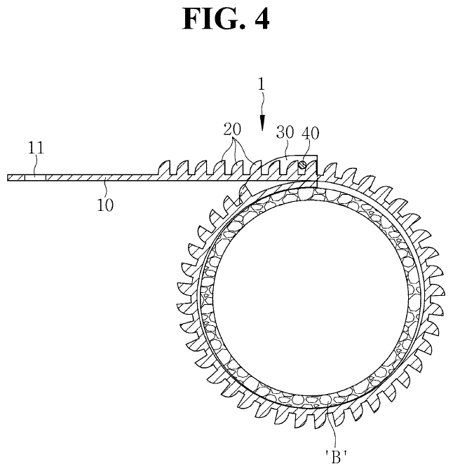

On an upper surface (outer surface) of the body portion 10, the plurality of engaging protrusions 20 that are caught by fixing wire 40 and maintain the diameter of the body portion 10 constant are arranged along a longitudinal direction of the body portion 10. The engaging protrusion 20 may be formed in a ratchet shape, and in particular, when the body portion 10 wraps around the blood vessel B, the engaging protrusions 20 may form an approximately circular shape and the engaging protrusion 20 may be formed in the ratchet shape having a curved front surface so as not to damage other body tissues. For example, as shown in FIG. 2 and FIG. 4, the engaging protrusion 20 may have a ratchet shape in which a front surface has a quarter-circular (¼-arc) profile and a rear surface is formed approximately perpendicular thereto.

The engaging protrusions 20 may be arranged at intervals equal to the diameter of the fixing wire 40, so that when the body portion 10 is rolled into a circular shape to wrap around the blood vessel B, the fixing wire 40 may be tightly inserted between the engaging protrusions 20, thereby maintaining a firm and stable fixing state.

The engaging protrusion 20 may be disposed in the center of the width of the body portion 10, and the width of the engaging protrusion 20 may be formed to be approximately ⅓ of the width of the body portion 10.

The fixing wire 40 is secured to each of two wing portions 30 that are formed to laterally protrude from opposite side edges of a rear end of the body portion 10. The fixing wire 40 may be made of a material that is safe for insertion into the human body and is not easily deformed physically or chemically, such as titanium.

The fixing wire 40 is disposed at a predetermined distance from an outer surface of the rear end of the body portion 10, such that after the front end of the body portion 10 and the engaging protrusions 20 pass under the fixing wire 40, a rear portion of one of the plurality of engaging protrusions 20 is caught and supported by the fixing wire 40, thereby preventing the body portion 10 from coming off to the outside of the fixing wire 40.

The wing portions 30, which fix both ends of the fixing wire 40, may have a shape in which the thickness gradually decreases from the rear toward the front so that, when the body portion is rolled into a circular shape, the overall configuration becomes substantially circular.

The vessel belt 1 may be applied as follows.

In a state where a lower surface (inner surface) of the body portion 10 is brought into contact with an outer surface of the blood vessel B, the front end of the body portion 10 is rolled into a circular shape and passed, from the rear side, between the rear end of the body portion 10 and the fixing wire 40. At this time, the ratchet-shaped engaging protrusions 20 formed on the outer surface of the body portion 10 have their inclined front surface naturally pass over the fixing wire 40.

When moving the body portion 10 in this manner, a tool may be hooked into the through hole 11 formed in the front end of the body portion 10, if necessary, to move the front end of the body portion 10.

When the body portion 10 is rolled to have a diameter corresponding to that of the blood vessel B and the movement of the body portion 10 is stopped, the rear surface of the engaging protrusion 20 positioned immediately in front of the fixing wire 40 is caught and supported by the fixing wire 40. Accordingly, the body portion 10 may maintain its rolled state and thus keep a constant diameter.

According to the present disclosure, the belt-shaped body portion 10 may be passed under the fixing wire 40 and rolled into a circular shape to wrap around the blood vessel B. When the movement is stopped at a diameter corresponding to the size of the blood vessel B, the circular shape may be maintained as it is. Therefore, the procedure becomes very simple and easy, and the diameter may be freely adjusted according to the size of the blood vessel B.

Although the detailed description of the present disclosure described above has been described with reference to preferred embodiments of the present disclosure, it will be understood by those skilled in the art or having ordinary knowledge in the art that various modifications and changes can be made to the present disclosure without departing from the spirit and technical scope of the present disclosure as set forth in the appended claims.

### EXPLANATION OF SYMBOLS

| | |
|---|---|
| B: Blood vessel | 1: Vessel belt |
| 10: Body portion | 11: Through hole |
| 20: Engaging protrusion | 30: Wing portion |
| 40: Fixing wire | |

1. A vessel belt comprising:
a body portion in the form of a band that wraps around a blood vessel;
a plurality of engaging protrusions arranged along a longitudinal direction on an outer surface of the body portion; and
a fixing wire having both ends fixed to opposite sides of a rear end of the body portion, the fixing wire being disposed at a predetermined distance from the outer surface of the body portion such that a front end of the body portion and the engaging protrusions pass thereunder, and after the front end of the body portion has passed, a rear portion of one of the plurality of engaging protrusions is caught and supported by the fixing wire.

2. The vessel belt according to claim 1, wherein two wing portions are formed to laterally protrude from opposite side edges of the rear end of the body portion, and both ends of the fixing wire are inserted into and fixed to the respective wing portions.

3. The vessel belt according to claim 1, wherein the fixing wire is made of titanium.

4. The vessel belt according to claim 1, wherein the front end of the body portion is shaped to be pointed forward.

5. The vessel belt according to claim 1, wherein a through hole for tool insertion is formed in the front end of the body portion.

6. The vessel belt according to claim 1, wherein each engaging protrusion is formed in a ratchet shape having a curved front surface, and the engaging protrusions are arranged at intervals equal to the diameter of the fixing wire.

7. The vessel belt according to claim 1, wherein a thickness of the body portion is formed to gradually decrease toward the front.

8. A vessel belt comprising:
a body portion made of a resin material in the form of a band that wraps around a blood vessel and has a front end formed to be pointed forward;
a plurality of engaging protrusions arranged along a longitudinal direction on an outer surface of the body portion, each engaging protrusion being formed in a ratchet shape having a curved front surface;
two wing portions formed to laterally protrude from opposite side edges of a rear end of the body portion; and
a fixing wire having both ends fixed to the wing portions, the fixing wire being disposed at a predetermined distance from the outer surface of the body portion so that the front end of the body portion and the engaging protrusions pass under the fixing wire, and after the front end of the body portion has passed, a rear portion of one of the plurality of engaging protrusions is caught and supported by the fixing wire.

9. The vessel belt according to claim 8, wherein the fixing wire is made of titanium.

10. The vessel belt according to claim 8, wherein a through hole for tool insertion is formed in the front end of the body portion.
